(19) United States
(12) Patent Application Publication
(10) Pub. No.: US 2018/0304057 A1
(43) Pub. Date: Oct. 25, 2018

(54) BALLOON MATERIAL FOR A BALLOON CATHETER AND PROCESS FOR PRODUCING THE BALLOON

(71) Applicant: BIOTRONIK AG, Buelach (CH)

(72) Inventor: Alwin Schwitzer, Buelach (CH)

(21) Appl. No.: 15/955,262

(22) Filed: Apr. 17, 2018

(30) Foreign Application Priority Data
Apr. 24, 2017 (EP) 17167689.3

(51) Int. Cl. A61M 29/02; A61L 29/06; B29C 49/04; B29C 49/00

(52) U.S. Cl. CPC A61M 29/02; A61L 29/06; B29K 2077/00; B29C 49/0005; A61M 2207/00; B29C 49/04

(57) ABSTRACT

A balloon for a balloon catheter has an inflatable, single-layer balloon envelope, which surrounds an interior and is dilatable by filling the interior with a fluid. The balloon envelope consists of polyamide 6.12 or includes or substantially consists of polyamide 6.12.

### PRIORITY CLAIM

This application claims priority under 35 U.S.C. § 119 and all applicable statutes and treaties from prior European Application EP 17167689.3, filed Apr. 24, 2017.

### FIELD OF THE INVENTION

This invention relates to a balloon, in particular a polymer for a balloon, a balloon catheter, and a process for producing the balloon.

### BACKGROUND

Balloons or balloon catheters are used, e.g., for expanding pathologically narrowed vessels in the body of a patient (balloon dilatation) or for the placement of vessel wall supports (so-called stents). Such balloons are disclosed, e.g., in US 2014/0116606 A1. This reference describes, e.g., a high-pressure balloon that consists of at least two layers (double membrane).

In theory, such a balloon can be produced, e.g., from a co-extruded tubing with two or more layers. It is also possible to produce such a multilayer balloon by pushing a corresponding number of tubes inside one another before forming the balloon. Furthermore, it is also possible to produce an outer balloon around an inner balloon in another way.

However, the previously mentioned processes are always comparatively elaborate. Furthermore, the greater total wall thickness of multilayer balloon envelopes makes them disadvantageous with respect to catheter placement at the destination (so-called deliverability).

### SUMMARY OF THE INVENTION

One aspect of the invention provides a balloon, and in particular a high-pressure balloon for a balloon catheter. The balloon has an inflatable, single-layer balloon envelope, which surrounds an interior and is dilatable by filling the interior with a fluid. The balloon envelope consists of polyamide 6.12 or includes polyamide 6.12. The balloon envelope can also include other substances, e.g., reinforcing substances, e.g., nanocomposites, to increase the balloon's burst pressure even more. Preferably, the balloon material is free of color or other additives which support imaging by a sonde. Preferably, the ratio of the thickness of the balloon envelope to the nominal balloon diameter is in the range from 5.0 μm/mm to 7.5 μm/mm, more preferably ˜6.3 μm/mm.

Another aspect of the invention provides a balloon catheter with:
an outer shaft with a distal end section;
an inner shaft guided in the outer shaft, the inner shaft having a distal end section that projects out beyond the distal end section of the outer shaft; and with
a balloon as above, wherein the fluid for dilating the balloon can be introduced through the outer shaft into the interior of the balloon envelope; and
a proximal end section of the balloon envelope being connected by material bonding with the distal end section of the outer shaft, and a distal end section of the balloon envelope being connected by material bonding with the distal end section of the inner shaft.

According to one embodiment, the balloon envelope is preferably laid in lengthways folds in undilated state, and nestles especially closely to the inner catheter.

One embodiment of the present balloon catheter further provides that the said material bonding connection(s) are produced by welding the proximal end section of the balloon envelope with the distal end section of the outer shaft, and/or by welding the distal end section of the balloon envelope with the distal end section of the inner shaft.

Finally, another aspect of this invention is the disclosure of a production process for a balloon for a balloon catheter, the balloon envelope of this balloon being dilatable by means of compressed air and being formed from a material that is polyamide 6.12 or that has polyamide 6.12.

One embodiment of the present process provides that the balloon envelope is produced by extruding the material into a single-layer tubular blank that surrounds an interior and that extends in an axial direction.

Here tubular means, in particular, that the blank has a wall around it, in particular a cylindrical wall that surrounds the interior of the blank, and two openings opposite one another, an edge area around the respective opening forming the proximal or distal end section of the balloon envelope to be produced.

A preferred embodiment of the process provides that the blank is stretched in the axial direction, e.g., by applying an axial stretching force to the extruded blank and by heating the extruded blank, so that the latter can stretch in the axial direction while the axial stretching force is maintained. After this, the blank is preferably cooled.

One embodiment of the process further provides that the extruded blank is brought to a temperature in the range from 120° C. to 160° C., especially 140° C., and is radially expanded to the shape of the balloon envelope.

One embodiment of the process further provides that the expansion comprises applying a pressure in the range from 20 bar to 50 bar, especially 30 bar to 35 bar, to the interior of the blank.

According to one embodiment, the expansion comprises putting the blank into a mold, heating the mold to the said temperature, and radially expanding the stretched tube using the said pressure in the interior of the blank.

After the balloon envelope has been formed, the mold is cooled and the balloon is removed.

### DESCRIPTION OF THE DRAWINGS

Further features and advantages of this invention are explained in the following description of the figures, which show a sample embodiment of the invention. The figures are as follows:

FIG. 1 a schematic sectional representation of a preferred balloon or balloon catheter; and

FIG. 2 a schematic representation of the production of a preferred balloon.

FIG. 3 the moisture absorption of polyamides (PA6, PA66, PA612, PA12);

FIG. 4 the tensile modulus of elasticity of polyamides (PA66, PA6, and PA12);

FIG. 5 shows the balloon compliance of a Grilamid 2D balloon compared with a conventional single-layer PA12-based high-pressure balloon;

FIG. 6 stress-strain curves (calculated from the compliance curves shown in FIG. 5 with the wall thicknesses of the molded balloons);

FIG. 7 the RBP of the tested balloons;

FIG. 8 the compliance of tested balloons;

FIG. 9 the balloon fatigue of tested balloons;

FIG. 10 the amide group (—HN—CO—) characteristic of polyamides, as is also present in peptides;

FIG. 11 a hydrogen bond (see arrow) connecting amide groups as shown in FIG. 10; these hydrogen bonds are responsible for the thermal and mechanical properties of polyamides. The binding energy of the hydrogen bonds is about 20 kJ/mol. The hydrogen bond is broken only under high loads, and is immediately reestablished after a displacement;

FIG. 12 shows the structure of PA66;

FIG. 13 shows the structure of PA6; and

FIG. 14 shows the structure of PA12.

### DETAILED DESCRIPTION

The substance polyamide 6.12 is poly(hexamethylene dodecanediamide), which has the CAS number 26098-55-5. That is, polyamide 6.12 is a polyamide with the monomer units hexamethylene diamine and 1,12-dodecanoic acid, in contrast to the state of the art balloon material polyamide 12, whose monomer unit is laurolactam.

The present balloon material polyamide 6.12 advantageously allows the production of a high-pressure balloon with a rated burst pressure (RBP) that is greater than 20 bar and has fatigue durability (20 cycles) at the RBP. Furthermore, the balloon material advantageously allows the balloon wall to be comparatively thin, so that the folded balloon has a small diameter and is suitable for an introducer system with correspondingly small French sizes. Furthermore, this allows better trackability. The balloon material additionally makes it simple to produce the starting tube (balloon tube) for forming the balloon. In particular, production does not require performing co-extrusion or pushing different tubes inside one another. Furthermore, the balloon forming can advantageously be made in one step on conventional balloon forming systems; double balloon forming is unnecessary. Finally, the present balloon material is advantageously weldable with the rest of the catheter components.

The fact that the present balloon envelope is in the form of a single layer means, in particular, that the balloon envelope is formed from single material layer that forms an outermost outer surface of the balloon envelope facing outward, and an innermost inner surface of the balloon envelope facing the interior.

The present material advantageously allows extrusion of the material, in particular for producing tubular blanks, and also allows thermoforming of such a blank to form balloons. Furthermore, a balloon produced in this way is sterilizable and is weldable with the rest of the catheter components, so that the high pressure properties of the balloon are also transferable to the entire catheter.

Advantageously and surprisingly, at these thicknesses, the present balloon, in particular high-pressure balloon, which comprises a dilatable and single-layer envelope, has an RBP greater than 20 bar and fatigue durability (20 cycles) at the RBP. The RBP refers to the pressure that is defined by the standards ISO 25539-1, ISO 25539-2, and ISO 10555-4. As defined by the FDA's “Guidance for Industry and FDA Staff Class II Special Controls Guidance Document for Certain Percutaneous Transluminal Coronary Angioplasty (PTCA) Catheters; Guidance for Industry and FDA”, the rated burst pressure (RBP) is the pressure that 99.9% of the balloons withstand without bursting at the 95% confidence level.

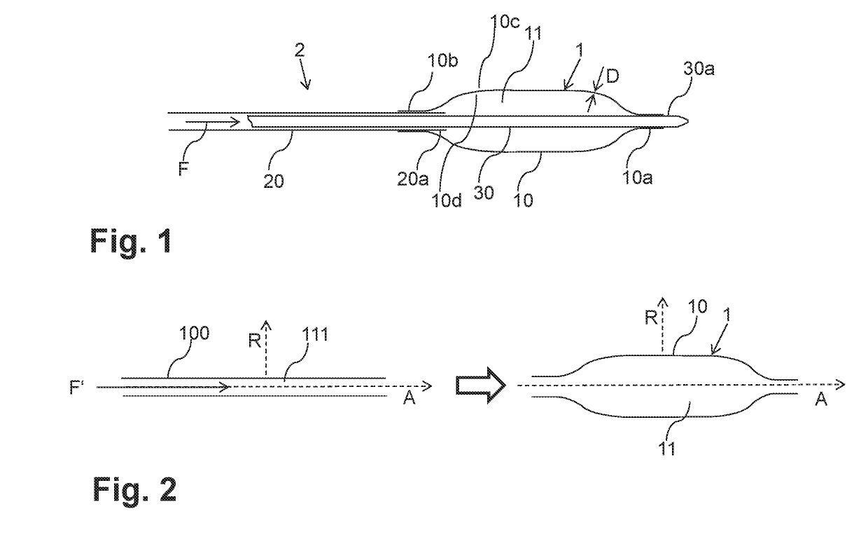

FIG. 1 is a schematic sectional view of a preferred balloon 1 or balloon catheter 2. Balloon 1 shows an inflatable and single-layer balloon envelope 10 that surrounds an interior 11 and can be dilated or inflated by filling the interior 11 with a fluid F. The balloon envelope 10 consists of polyamide 6.12 or includes or substantially consists of polyamide 6.12, and can also contain reinforcing substances such as nanocomposites.

Preferably, the ratio of the balloon's thickness to the nominal balloon diameter is in the range from 5.0 μm/mm to 7.5 μm/mm, preferably ˜6.3 μm/mm.

According to one aspect of the invention, the balloon 1 is used as a balloon of a balloon catheter 2. The latter has an outer shaft 20 with a distal end section 20a, and an inner shaft 30 that is guided in the outer shaft 20 and that has a distal end section 30a that projects out beyond the distal end section 20a of the outer shaft 20. A proximal end section 10b of the balloon envelope 10 is now connected by material bonding with the distal end section 20a of the outer shaft 20, and a distal end section 10a of the balloon envelope 10 is connected by material bonding and fluid-tight with the distal end section 30a of the inner shaft 30. This can involve introducing a fluid F to dilate the balloon 1 through the outer shaft (past the inner shaft) into the interior 11 of the balloon envelope 10. The said connections by material bonding are preferably in the form of welded connections.

FIG. 2 is a schematic of a preferred process for making the balloon 1. This involves first producing the balloon envelope 10 by extruding the material formed by polyamide 6.12 or having polyamide 6.12 into a single-layer tubular (e.g., cylindrical) blank 100 that surrounds an interior 111 and that extends along an axial direction A. Before the balloon forming, the blank 100 can undergo stretching in the axial direction A (e.g., by heating and applying a stretching force acting in the axial direction A).

The possibly stretched blank 100 is then brought to a temperature in the range from 120° C. to 160° C., especially 140° C., and is expanded in the radial direction R into the shape of the balloon envelope 10. The expansion is preferably done by applying a pressure in the range from 20 bar to 50 bar, especially 30 bar to 35 bar, to the interior 111 of the blank 100, e.g., by introducing a fluid F (e.g., compressed air) with a corresponding pressure into the interior 111 or 11 (also see above).

The polyamide 6.12 used can be, e.g., Grilamid 2D 20 of the manufacturer EMS-CHEMIE AG or Vestamid® D16, Vestamid® D18, Vestamid® D22, or Vestamid® D26 of the manufacturer EVONIK Industries or other manufacturers.

According to one example of the invention, a single-layer balloon tube has been extruded from Grilamid® 2D 20. The balloon forming is done at a temperature of ˜140° C. with a blowing pressure of ˜30 bar on a conventional balloon forming system, which provides, e.g., forming the balloon in a closed mold with the application of pressure from the inside.

The balloon produced in this way from Grilamid 2D 20 has a diameter of 3.0 mm and achieves an RBP of 30 bar with a wall thickness D of 0.019 mm (6.3 μm/mm).

By contrast, a comparison balloon made of polyamide 12 (PA12) achieved an RBP of 24 bar with twice the wall thickness (0.040 mm). That is, the balloon produced according to the invention with a 5% [sic] smaller balloon wall thickness D has a burst pressure (RBP) that is 6 bar higher at the balloon stage.

Furthermore, because the polymer used according to the invention—polyamide 6.12—is produced from a diamine and a dicarboxylic acid, it can advantageously show no blooming effect compared with PA12 or polymers derived from it (e.g., PEBAX® types). The reason why is that lactam-based polyamides, e.g., polyamide 12, can have unreacted lactam in the polymer that migrates, over time, from the formed part to the surface. In the case of polyamides that are formed from dicarboxylic acids and diamines this is impossible, since any possibly unreacted monomers in the polymer matrix are in the form of a salt.

In addition, the present balloons can easily be produced in a cost-effective manner from a single-layer extruded tube.

Other examples of the invention and comparison tests are presented in detail below, and these substantiate the advantageous suitability of polyamide 6.12 as a balloon material or a component of a balloon.

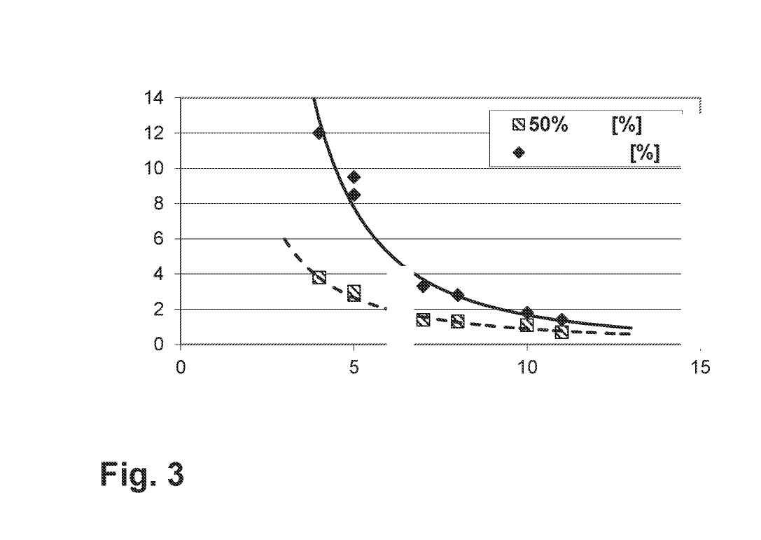

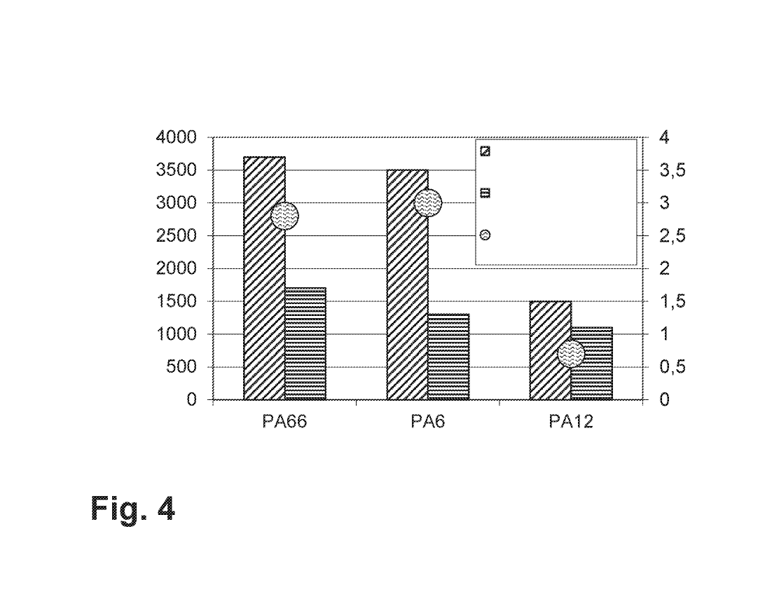

Relating to this, the reasons why polyamide 6.12 is especially suitable as a balloon material also include its comparatively low moisture absorption (FIG. 3: PA6/12 8 C atoms, PA6, PA66: 5 C atoms). This is significant since polyamides with higher moisture absorption exhibit a stronger decrease in mechanical properties. Relating to this, FIG. 4 shows the tensile modulus of elasticity of other polyamides (PA66, PA6, and PA12).

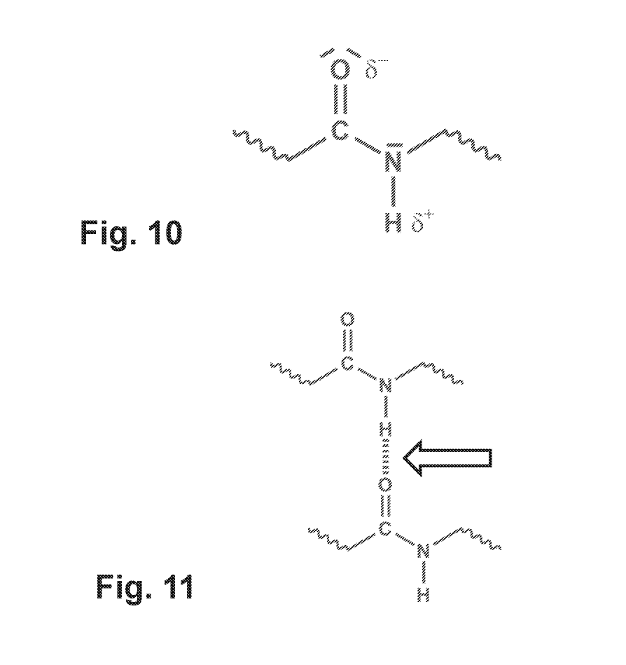

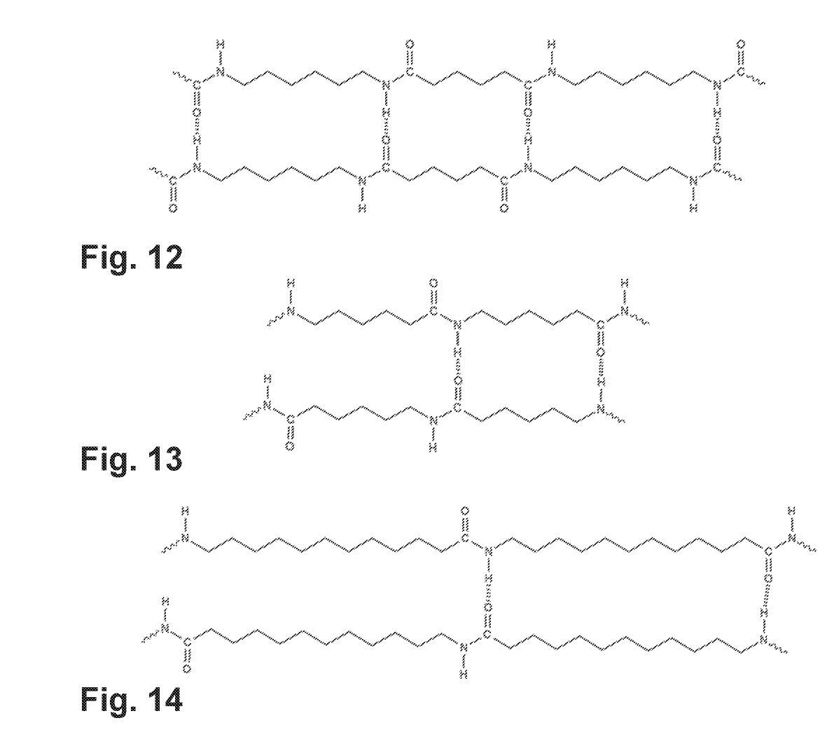

The more carbon atoms are present between the amide bonds (see, e.g., FIGS. 10 through 14), the lower the moisture absorption of the polyamide in question. Furthermore, however, as the number of carbon atoms between the amide bonds increases, the tensile modulus of elasticity and thus the mechanical strength/stiffness of the polyamide in question decreases.

Therefore, polyamide 6.12 is an advantageous alternative to the existing polyamides (cf. FIG. 3) for balloons, especially vascular catheters, since the comparatively low moisture absorption is accompanied by a correspondingly small reduction in the mechanical properties due to the moisture absorption, in particular its modulus of elasticity is higher than that of polyamide 12 (PA12).

The polyamide 6.12 discussed below is, in particular, Grilamid 2D 20 (EMS-Chemie) or Vestamid® D/Evonik.

As has already been explained above, polyamide 6.12 (PA612) is a polyamide that can be produced from 1,6-hexanediamine and 1,12-dodecanoic acid through condensation polymerization. This is in contrast to polyamide 12, which is produced from laurolactam, polyamide 6, which is produced from caprolactam, or polyamide 66, which is produced by condensation polymerization from 1,6-hexanediamine and adipic acid.

In comparison with PA12 PEBAX® 7033 and PA6/12 (copolyamide from caprolactam and laurolactam, properties depend on the ratio of the two monomers), polyamide 6.12 shows the following properties:

| | Grilamid ® | Grilamid ® | PEBAX ® | PA6/12 |
|---|---|---|---|---|
| | 2D 20 | L25 | 7033 | Copolyamide |
| Modulus | 1,600 | 1,100 | 384 | 400-550 |
| of elasticity | | | | |
| conditioned | | | | |
| (50% RM)/MPa | | | | |
| Tensile strength | 50 | 50 | 54 | |
| @ break/MPa | | | | |
| Elongation | 16 | >50 | >350 | |
| @ break/% | | | | |
| Shore D hardness | 81 | 70 | 61 | |
| Polymer | PA612 | PA12 | PEBA | PA6/12 |
| classification | | | | |

In particular, after conditioning (water absorption by storage at 50% relative humidity) the polyamide PA612 (Grilamid® 2D 20) shows a higher modulus of elasticity than PA12 or PEBAX® 7033, which are used in the prior art as single-layer balloon materials for catheters.

As was already explained, PA612 is synthesized from a diamine and a dicarboxylic acid, so this polyamide cannot (in contrast to PA12 or the PA12-based PEBAX® types) have any free monomer that could migrate out of the component due to ageing (so-called blooming).

Furthermore, as an example a single-screw extruder was used to extrude, from polyamide 612 (Grilamid® 2D 20, EMS Chemie), a single-layer tube, which was used to produce balloons in a balloon forming system by conventional stretch blow molding using similar temperatures as for tubes made from PA12 or PEBAX® (with a blowing pressure of ˜30 bar). The PA612 balloons were then used to produce PTCA catheters with existing designs, the properties of the balloons/catheters (with a diameter of ˜3.0 mm and a length of 20 mm) being compared with those of a single-layer high-pressure balloon catheter (whose balloon material was PA12).

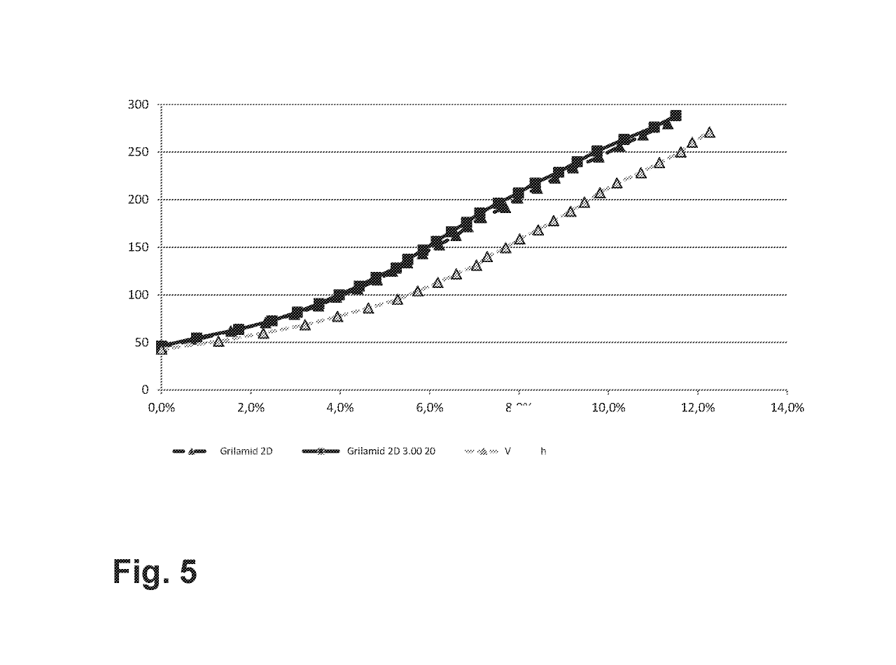

With respect to balloon compliance (the change in the balloon diameter as a function of the dilatation pressure), which is shown in FIG. 5, the following picture results. The compliance of the Grilamid® 2D 20 balloons is in the range of ˜10 bar to ˜20 bar, which is comparable with an existing 1-layer high-pressure balloon (PA12 as balloon material) with smaller balloon wall thickness.

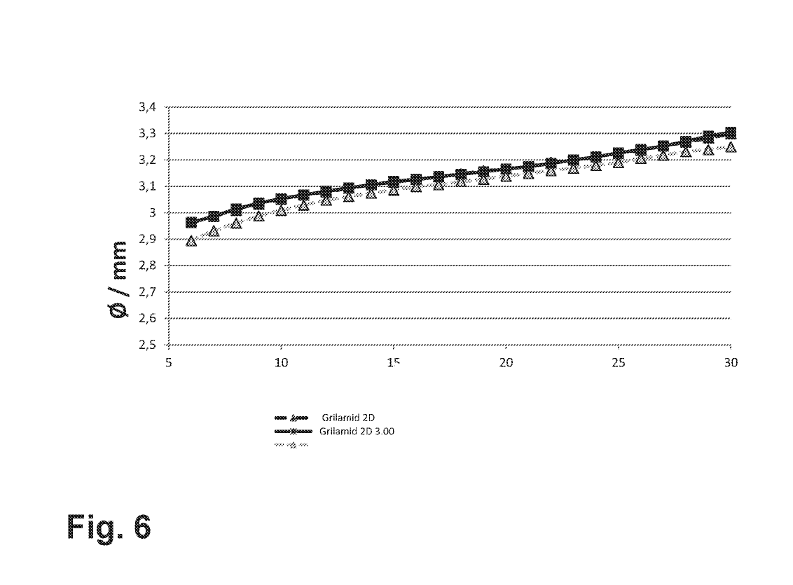

The higher strength of Grilamid 2D 20 balloons than that of an existing single-layer (PA12) balloon is also apparent in the stress-strain diagram, which is shown in FIG. 6. In particular, this diagram shows that the radial stretching of the Grilamid® 2D 20 balloons requires a higher stress (pressure 1.2 to 1.4 times greater) than do existing 1-layer PA12 high-pressure balloons.

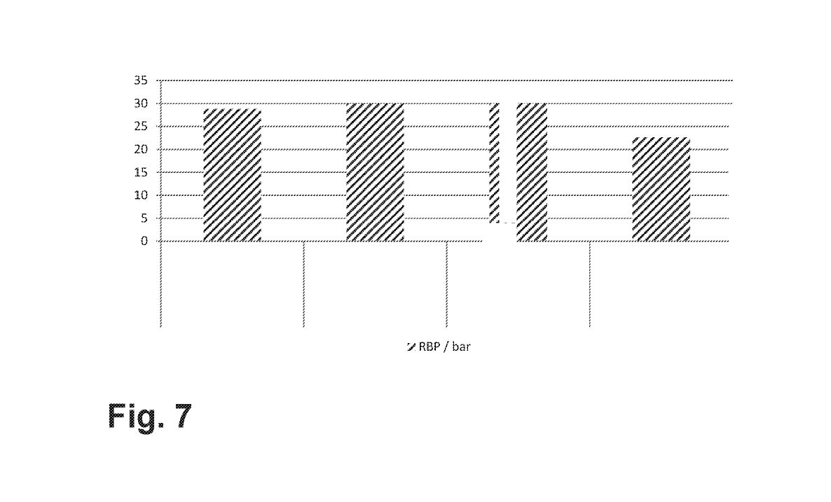

With regard to the rated burst pressure (RBP), i.e., the permissible [sic] burst pressure, FIG. 7 shows that it is higher for the Grilamid® 2D components than it is for the comparison balloon (a conventional PTCA balloon 3.0/30: 17 bar).

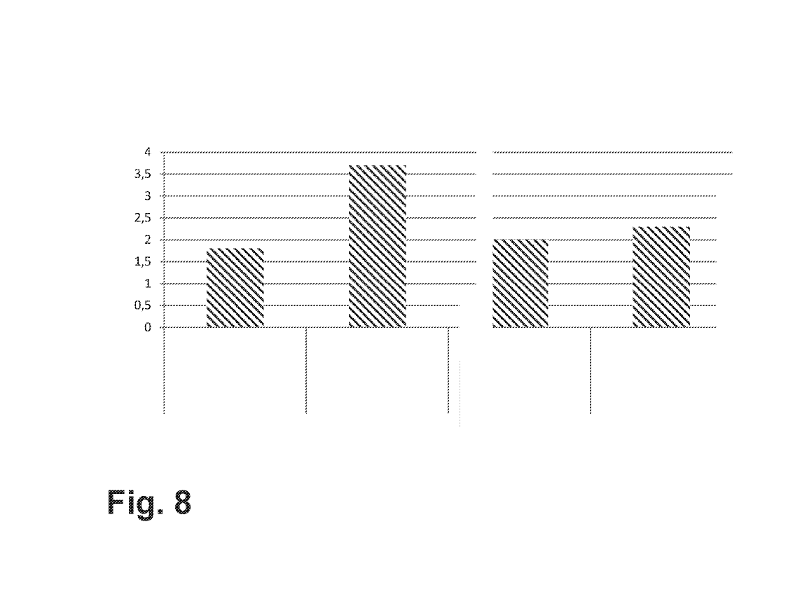

The compliance of the Grilamid 2D components shown in FIG. 8 also has values that are appealing in comparison with the conventional PTCA balloon: 5.0%.

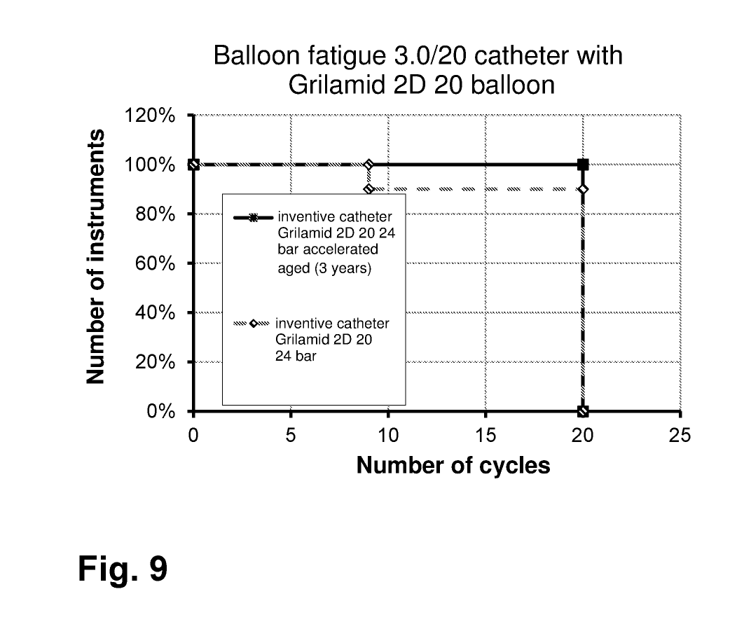

As can also be inferred from FIG. 9, which shows balloon fatigue [sic], i.e., the durability of the balloon in question under repeated load, the material Grilamid® 2D passes the balloon fatigue test, which also proved the tightness of the welding of the balloons with the inner shaft, outer shaft, and tip of the catheter.

The summary of results with Grilamid 2D 20 (PA 612) balloons at the balloon stage (properties of the balloons after balloon forming) gives the following picture:

| Balloons | RBP | Wall thickness D/μm |
|---|---|---|
| Grilamid ® 2D 20 balloons | 30.0 bar | 19 |
| Comparison | 24 bar | 20 |
| single-layer high-pressure balloon, | | |
| PA12-based | | |
| PEBAX ® MED balloons | 25 bar | 24 |

Thus, the present balloon material Grilamid 2D with a thinner double wall thickness can achieve a higher RBP than PA12 or PEBAX® balloons. The balloon properties are preserved even after being sterilized twice.

In summary, at the catheter stage (balloon properties on the final sterilized catheter) the following picture results (cf. FIG. 9):

| Compliance/RBP | Compliance/RBP | Fatigue @ 24 bar/20 cycles withstood | |
|---|---|---|---|
| 0-time | acc aged (2Y) | bar 0-time | acc aged (2 years) |
| 2.40%/25.1 bar | 2.30%/23.9 bar | 9/10 | 10/10 |

Stent dislodgement force

| 0-time | acc aged (2 years) |
|---|---|
| 5.8N | 6.2N |

Thus, the result here is low compliance of 2.4%; balloon fatigue [sic] (20 cycles) could be demonstrated at 24 bar. Furthermore, there is sufficient stent dislodgement force.

It will be apparent to those skilled in the art that numerous modifications and variations of the described examples and embodiments are possible in light of the above teaching. The disclosed examples and embodiments may include some or all of the features disclosed herein. Therefore, it is the intent to cover all such modifications and alternate embodiments as may come within the true scope of this invention.

What is claimed is:

1. A balloon for a balloon catheter, the balloon comprising an inflatable, single-layer balloon envelope, which surrounds an interior and is dilatable by filling the interior with a fluid, wherein the balloon envelope consists of polyamide 6.12 or comprises polyamide 6.12.

2. A balloon according to claim 1, wherein a ratio of the thickness of the balloon envelope to the nominal balloon diameter is in the range from 5.0 μm/mm to 7.5 μm/mm.

3. A balloon catheter, comprising:
an outer shaft with a distal end section;
an inner shaft guided in the outer shaft and with a distal end section that projects out beyond the distal end section of the outer shaft; and
a balloon according to claim 2, wherein the outer shaft and balloon are configured such that the fluid for dilating the balloon can be introduced through the outer shaft into the interior of the balloon envelope; wherein
a proximal end section of the balloon envelope is connected by material bonding with the distal end section of the outer shaft, and a distal end section of the balloon envelope is connected by material bonding with the distal end section of the inner shaft.

4. A balloon catheter according to claim 3, wherein the proximal end section of the balloon envelope is welded with the distal end section of the outer shaft.

5. A balloon catheter according to claim 4, wherein the distal end section of the balloon envelope is welded with the distal end section of the inner shaft.

6. A balloon catheter according to claim 3, wherein the distal end section of the balloon envelope is welded with the distal end section of the inner shaft.

7. A balloon catheter, comprising:
an outer shaft with a distal end section;
an inner shaft guided in the outer shaft and with a distal end section that projects out beyond the distal end section of the outer shaft; and
a balloon according to claim 1, wherein the outer shaft and balloon are configured such that the fluid for dilating the balloon can be introduced through the outer shaft into the interior of the balloon envelope; wherein
a proximal end section of the balloon envelope is connected by material bonding with the distal end section of the outer shaft, and a distal end section of the balloon envelope is connected by material bonding with the distal end section of the inner shaft.

8. A balloon catheter according to claim 7, wherein the proximal end section of the balloon envelope is welded with the distal end section of the outer shaft.

9. A balloon catheter according to claim 8, wherein the distal end section of the balloon envelope is welded with the distal end section of the inner shaft.

10. A balloon catheter according to claim 7, wherein the distal end section of the balloon envelope is welded with the distal end section of the inner shaft.

11. A balloon according to claim 1, wherein the balloon catheter substantially consists of polyamide 6.12 and includes a reinforcing nanocomposite.

12. A production process for a balloon for a balloon catheter, the process comprising: providing material that consists of polyamide 6.12 or comprises polyamide 6.12; and extruding the material into a single-layer tubular blank that surrounds an interior and that extends in an axial direction.

13. A process according to claim 12, wherein the blank is stretched in the axial direction.

14. A process according to claim 13, further comprising heating the extruded blank to a temperature in the range from 120° C. to 160° C. and radially expanding the blank into the shape of a balloon envelope.

15. A process according to claim 14, further comprising, during said radially expanding, applying a pressure in the range from 20 bar to 50 bar to the interior of the blank.
